(19) United States
(12) Patent Application Publication
(10) Pub. No.: US 2010/0291155 A1
(43) Pub. Date: Nov. 18, 2010

(54) ALLERGY VACCINE COMPOSITION FOR MUCOSAL ADMINISTRATION

(75) Inventor: Maria Mercedes Ferreras Gomez, Kgs. Lyngby (DK)

Correspondence Address: BIRCH STEWART KOLASCH & BIRCH, PO BOX 747, FALLS CHURCH, VA, 22040-0747, US

(73) Assignee: ALK-Abello A/S, Horsholm (DK)

(21) Appl. No.: 12/812,027

(22) PCT Filed: Jan. 7, 2009
(86) PCT No.: PCT/EP2009/050137

§ 371 Date: Jul. 7, 2010

Related U.S. Application Data
(60) Provisional application No. 61/019,792, filed on Jan. 8, 2008.

(30) Foreign Application Priority Data
Jan. 8, 2008 (DK) PA 2008 00026

(51) Int. Cl. A61K 39/35; A61P 37/08

(52) U.S. Cl. 424/275.1

(57) ABSTRACT

The present invention relates to an allergy vaccine composition for mucosal administration comprising a cysteine protease allergen in a reduced active state or in an oxidised inactive state, The inventions further relates to an adjuvant system for use in a vaccine for mucosal administration comprising a cysteine protease.

### TECHNICAL FIELD

The present invention relates to an allergy vaccine composition for mucosal administration comprising a cysteine protease allergen.

### BACKGROUND OF THE INVENTION

Allergy is major health issue worldwide. The atopic diseases, such as allergic rhinitis, asthma, and atopic eczema, have become one of the primary causes of chronic bad health. Nowadays, the term ‘allergy’ is often connected to IgE mediated diseases. Everyone is subjected to possible allergens every day. Many of the allergens are small, high soluble proteins carried on dry particles such as cat dander, house dust mites' faeces, or pollen grains. When we breath, these particles encounter the airway mucosa.

Some people are hereditary predisposed for producing an immediate hypersensitivity reaction against allergens. This is called atopy and atopic individuals have a higher level of eosinophils and a higher total level of IgE in their circulation than non-atopic individuals, They also often have more than one atopic disease.

Exposure to allergens can result in an overreaction of the immune system called an allergic reaction or hypersensitivity reaction. Hypersensitivity reactions are divided into four categories: the type I hypersensitivity is an immediate type of hypersensitivity reaction mediated by IgE and is mostly the cause of allergic rhinitis, asthma, and systemic anaphylaxis. When exposed to allergens, non-atopic individuals will only mount a weak immunological response producing the allergen-specific IgG1 and IgG4 antibodies (Th1 response). The atopic individuals, however, respond to allergens as it being a pathogen. The B cells are thus stimulated into producing IgE instead of producing IgG (Th2 response). IgE then binds to the mast cells through the high-affinity IgE receptor, FcεRI, The second time the individual encounters the same allergen, it results in a hypersensitivity reaction. Two mast cell bound IgE molecules cross-link with a single allergen, stimulating the release of the toxic mediators such as histamine and heparin, cytokines, and enzymes from the granules of the mast cell. The release of histamine results in an increased vascular permeability, vasodilation (dilation of local blood vessels) and smooth muscle contraction. All these factors combined result in an allergic reaction. The effect on the airways are wheezing, coughing, and sneezing and in the gastrointestinal tract it will often cause diarrhea and vomiting.

The allergic response mediated by the allergens from the house dust mite is type I hypersensitivity. In humid areas of the world, dust mites are everywhere, Up to 13 different species have been identified living in house dust, and 80% of all these mites are represented by the three most common HDM species Dermatophagoides pteronyssinus, Dermatophagoides farinae, and Euroglyphus maynei. In these warm geographic areas, dust mite species account for positive skin test reactions in almost 30% of the population. HDMs are considered to be one of the main causes to allergy.

The mite faeces and mite bodies are the sources of the allergens provoking allergic responses. The size of the mite faecal pellet is between 10-20 μm which makes it easily airborne. To date 14 different groups of allergens from D. pteronyssinus also known as the European house dust mite—are identified.

In a group of mite allergic individuals Der p 1 and Der p 2 will bind to IgE from the sera in over 60% of the individuals. They are therefore considered major allergens.

Der p 1

Der p 1 is a major house dust mite allergen and is considered one of the most immunodominant allergens when it comes to dust mite specific IgE mediated hypersensitivity. It is a 25371 Da cysteine protease and belongs to clan CA—the papain-like cysteine proteases. It is synthesized as an inactive precursor of 320 amino acids. In order for the protein to become mature, a cleavage of the pro-peptide is necessary, becoming a 222 amino acids-long protein. Der p 1 has only been found in its mature form in the D. pteronyssinus faecal particles, and therefore it is the mature protein which constitutes the allergen.

The catalytic activity of the papain-like cysteine proteases are dependent on three residues known as the catalytic triad. These are a cysteine, a histidine, and an asparagine. The catalytic cysteine must by reduced in order for the enzyme to be active. The cysteine proteases can be irreversibly inhibited by E64 (L-trans-epoxysuccinyl-leucylamido(4-guanidino)butane).

Human in vitro studies have shown that Der p 1 can cleave a number of proteins leading to an enhanced Th2 response. Furthermore, in vivo studies in mice have shown that when sensitized mice were intranasally exposed to proteolytically active Der p 1, the total IgE level in the circulation became significantly higher, as opposed to mice exposed to Der p 1 irreversibly inactivated with E64. Also the lung inflammation was notably higher for mice exposed to the proteolytically active Der p 1.

These studies indicate that Der p 1 proteolytic activity might play a role in eliciting an allergic response.

### Prior Publications

Wan et al. (The Journal of Clinical Investigation, July 1999, Volume 104, Number 1, 123-133) discloses an in vitro study of the role of Der p 1 in transepithelial delivery, and it was found that Der p 1 causes disruption of intercellular tight junctions, which are the principal components of the epithelial paracellular permeability barrier. Specifically, it was found that Der p 1 led to cleavage of the tight junction adhesion protein occluding in confluent airway epithelial cells. Tight junction breakdown non-specifically increased epithelial permeability allowing Der p 1 to cross the epithelial barrier and to reach dendritic antigen-presenting cells. It is speculated that this role of Der p 1 may be the initial step in development of asthma.

Kauffman et al. (Clinical and Molecular Allergy 2006, 4:5) discloses the same findings as Wan et al. mentioned above. It is further mentioned that reduction of natural Der p 1 with gluthathione produced its most active reduced form.

Takai et al. (Int Arch Allergy Immunol 2005; 137:194-200) discloses a system of preparing correctly folded active recombinant Der p 1 and Der f 1 and a study of their proteolytic activity, which is indicated to have importance in the pathogenesis of allergy. It is mentioned that the recombinant molecules prepared are activated with reducing agents, such as DTT and L-cysteine. Reference is made to an earlier study, wherein it is speculated that cysteine protease activity of natural Der p 1 could be activated in vivo by glutathione.

The object of the present invention is to provide an improved allergy vaccine composition.

### SUMMARY OF THE INVENTION

A first aspect of the present invention relates to an allergy vaccine composition for mucosal administration comprising a cysteine protease allergen in a reduced active state. The first aspect of the present invention also relates to an allergy vaccine composition for mucosal administration comprising a cysteine protease allergen in an oxidised inactive state.

In the first aspect, the present invention is based on the recognitions 1) that in the use of a cysteine protease allergen as an active substance in an allergen vaccine for mucosal administration, the issue of whether the cysteine protease allergen is in its reduced active or its oxidised inactive state is an important issue, because active cysteine protease has an enhanced immunological activity and hence potency in connection with mucosal administration and 2) that it is in fact possible and advantageous to control the reduction/oxidation state of the cysteine protease in an allergen vaccine for mucosal administration. Thus, by controlling the reduction/oxidation state of the cysteine protease it is possible to control more precisely the immunological activity and hence the potency of the vaccine. In conventional allergy vaccines the reduction/oxidation state of cysteine proteases is not controlled and hence there is a risk that the reduction/oxidation state and hence the potency of the vaccine may vary. In particular the present invention has provided a possibility to prepare an allergy vaccine for mucosal administration with enhanced immunological activity and potency by using active cysteine protease. If desired, inactive cysteine protease may be used, which may be desired for some applications, e.g. for very potent allergens, or in order to reduce the risk of side effects, such as itching, in connection mucosal administration.

The first aspect of the invention is further based on the recognition that addition of glutathione to the allergy vaccine composition will result in an enhancement of the immunological activity and potency of the vaccine, since it will stimulate transformation of oxidised inactive cysteine protease to active reduced cysteine protease or ensure that reduced active cysteine protease is not transformed to oxidised inactive cysteine protease. Thus, in vivo the oxidation/reduction state of a cysteine protease molecule is determined by the oxidation/reduction state of the microenvironment of the molecule. Thus, when the vaccine is administered to the mucosa of a patient, the cysteine protease might well be exposed an oxidising microenvironment, and the effect of such an oxidising microenvironment may be prevented or decreased by the local presence of glutathione, which has a reducing effect.

Additionally, the first aspect of the invention is based on the experimental finding that gluthathione S-transferase has an unexpected strong catalysing effect on glutathione reducing activity on cysteine protease. Further, it is based on the recognition that, like for the addition of glutathione, see above, the addition of glutathione S-transferase to the allergy vaccine composition will result in an enhancement of the immunological activity and potency of the vaccine, since it will stimulate transformation of oxidised inactive cysteine protease to active reduced cysteine protease or ensure that reduced active cysteine protease is not transformed to oxidised inactive cysteine protease.

A second aspect of the invention relates to an adjuvant system for use in a vaccine for mucosal administration comprising a cysteine protease. The second aspect of the invention further relates to an antigen vaccine composition for mucosal administration comprising an antigen and an adjuvant system according to the invention.

The second aspect of the invention is based on the recognition that a cysteine protease can be used as an adjuvant in a vaccine composition, because a cysteine protease as explained above is assumed to play a role in eliciting an immune response by means of its proteolytic activity. Furthermore, this aspect of the invention is based on the recognitions that the use of cysteine protease as adjuvant may be enhanced by including glutathione alone or in combination with glutathione S-transferase so as to optimise the in vivo concentration of cysteine protease in its reduced state.

### SHORT DESCRIPTION OF THE FIGURES

FIG. 1: Proteolytic activity of purified nDer p 1, both in pool 1 and 2, in the presence or absence of the cysteine protease inhibitor E64. The activity of Der p extract was evaluated as well as control.

FIG. 2: Activation of nDer p 1 by GSH. The figure shows the time-courses of the activation of nDer p 1 by increasing concentrations of GSH

FIG. 3: Close-up of FIG. 2.

FIG. 4: Activation of nDer p 1 by GSH: The initial velocity of the progress curves of nDer p 1 activity is plotted against the GSH concentration used for the activation.

FIG. 5: Ability of nDer p 8 to catalyse the activation of Der p 1 by GSH. The activity was tested with 0.1 mM GSH and different concentrations of nDer p 8.

FIG. 6: Activation of Der p 1 by Der p 8 and GSH. The initial velocity of the progress curves are plotted against the nDer p 8 concentrations.

### DETAILED DESCRIPTION OF THE INVENTION

### Cysteine Protease

Cysteine proteases are proteases, wherein the nucleophile is the sulfhydryl group of a Cys residue. The first clearly recognised cysteine protease was papain. Crystal structures of papain and some closely related cysteine proteases have been determined. Cysteine proteases resembling papain are called papain-like cysteine proteases. It is believed that papain-like cysteine proteases share a Cys, His dyad of the catalystic site. Papain-like cysteine proteases are classified in a CA clan and a CC clan (viral papain-like cysteine proteases), wherein the CA clan comprises families C1, C2, C10, C12 and C19, and wherein clan CC comprises families C6, C7, C8, C9, C16, C21, C23, C27, C28, C29, C31, C32, C33, C34, C35, C36, C41, C42 and C43. Specific papain-like cysteine proteases are listed e.g. in Handbook of Proteolytic Enzymes edited by Alan J. Barrett et al., Academic Press, 1998, to which reference is made. The cysteine protease of the vaccine composition of the present invention is a papain-like cysteine protease of clan CA or of clan CC.

The three-dimensional structure of proDer p 1 corresponds closely to the three-dimensional structure of mature Der p 1. This fact is evidenced e.g. by comparisons of the structure of proforms and mature forms of various cysteine proteases. The crystal structures of two homologous cysteine proteases, caricain and cathepsin K, have been determined for both the pro and mature forms of these proteins. The structure of the mature region within the proform is virtually identical to that of the mature form in both cases (Groves et al., Structure, 1996, vol. 4, pp 1193 and LaLonde et al., Biochemistry, 1999, vol. 38, pp 862).

### Cysteine Protease Allergen

The cysteine protease allergen according to the present invention is a cysteine protease as defined elsewhere in this specification, which is also an allergen as defined elsewhere in this specification. The allergen may be an inhalant allergen, i.e. an airborne allergen, which may come into contact with the mucosa of the airway system of an individual. The allergen may also be a food allergen, which may come into contact with the mucosa of the digestive system of an individual.

In a particular embodiment of the invention, the cysteine protease allergen is selected from the group consisting of Ale o 1, Aca s 1, Blo t 1, Der f 1, Der p 1, Der m 1, Der s 1, Eur m 1, Gly d 1, Lep d 1, Pso o 1, Sui m 1 and Tyr p 1, in particular Der p 1.

In one embodiment of the invention the allergy vaccine composition for mucosal administration comprises a cysteine protease allergen in a reduced active state. It is believed that cysteine protease allergen in a reduced active state has an enhanced immunological activity and potency in mucosal administration, as the proteolytic activity of the allergen cleaves the tight junction of the mucosal tissue hence allowing the allergen to cross the mucosa and contact the dendritic antigen-presenting cells. Such enhanced immunological activity and potency will potentially enhance the therapeutic effect of the allergy vaccine. Such enhanced therapeutic effect of the vaccine may be used to reduce the dose of the allergen in the vaccine and hence to reduce any undesired side effect, such as itching for sublingual vaccines, or to increase the therapeutic effect of the vaccine.

In a particular embodiment of the invention, 70% of the cysteine protease allergen, preferably 80%, more preferably 90%, more preferably 95%, more preferably 98% is in a reduced active state.

In another embodiment of the invention the allergy vaccine composition for mucosal administration comprises a cysteine protease allergen in an oxidised inactive state. For some vaccine formulations, routes of administration and types of allergens, it is preferred to use cysteine protease allergen in an oxidised inactive state to moderate the level of immunological activity and potency with a view to control the level of side effects.

In a particular embodiment of the invention, 70% of the cysteine protease allergen, preferably 80%, more preferably 90%, more preferably 95%, more preferably 98% is in an oxidised inactive state.

The cysteine protease of the vaccine composition of the invention may be obtained by any conventional method, including recombinant techniques and purification from biological materials. The cysteine protease allergen of the invention may be in the form of an allergen extract, a purified fraction of an allergen extract, a modified allergen, a recombinant allergen or a mutant of a recombinant allergen with at least some proteolytic activity. An allergenic extract may naturally contain one or more isoforms of the same allergen, whereas a recombinant allergen typically only represents one isoform of an allergen. The mutant allergen may be a low IgE-binding mutant, e.g, a low IgE-binding allergen according to WO 99/47680, WO 02/40676 or WO 03/096869 A2. In a preferred embodiment of the invention, the allergen composition is an allergen extract, a purified fraction of an allergen extract or a recombinant allergen.

Reduced cysteine protease of the vaccine composition of the invention may be obtained by treating cysteine protease with any suitable reducing agent, including inorganic, organic and biological reducing agents. It is preferred to use a pharmaceutically acceptable reducing agent, such as cysteine, glutathione, a combination of glutathione and glutathione S-transferase, Dithiothreitol (DTT), cysteamine, thioredoxin, N-acetyl-L-cysteine (NAC), alpha-lipoic acid, 2-mercaptoethanol, 2-mercaptoethanesulfonic acid, mercapto-propionyglycine or tris(2-carboxyethyl)phophine (TCEP).

Oxidised cysteine protease of the vaccine composition of the invention may be obtained by treating cysteine protease with any suitable oxidising agent, including inorganic, organic and biological oxidising agents. It is preferred to use a pharmaceutically acceptable oxidising agent, such as glutathione disulfide (GSSG), cystine or cystamine.

### Glutathione (GSH)

Glutathione is a tripeptide (C10H17N3O6S, CAS NO. 70-18-8), which is formed from L-cysteine, L-glutamate and glycine. Glutathione is found in many animals, wherein it is mostly present in reduced form. In healthy cells and tissue more than 90% of the total glutathione poll is in the reduced form (GSH) and less than 10% exists in the active form (GSSG). Glutahione is present in the human airway system in the lung fluid in an extracellular concentration of between 100 and 500 mM. Reduced glutathione serves as an antioxidant. Most eukaryotes and some bacteria are capable of synthesizing glutathione. In the reduced state, the thiol group of cysteine is able to donate and electron (H+) to other unstable molecules, such as reactive oxygen species. In donating an electron, glutathione itself becomes reactive, but readily reacts with another reactive glutathione to form glutathione disulfide (GSSG), GSH can be regenerated from GSSG by the enzyme glutathione reductase.

The glutathione of the vaccine composition of the present invention may be glutathione or a derivative thereof having the same reducing activity on cysteine protease.

The glutathione of the vaccine of the present invention may be prepared by any conventional method by which glutathione is obtainable, including chemical synthesis, enzymatic synthesis and purification from biological material. The glutathione of the vaccine of the present invention is in its reduced form (GSH) in combination with reduced cysteine protease. When a vaccine composition comprising a combination of reduced cysteine protease and reduced glutathione is administered to an individual, the inclusion of reduced glutathione in the vaccine composition will ensure that the level of reduced glutathione in the microenvironment surrounding the reduced cysteine protease will be high, and that accordingly that the reduced cysteine protease will stay in its reduced state. In the absence of glutathione a proportion of the cysteine protease will be subjected to oxidative surroundings and hence be transformed to oxidised cysteine protease.

### Pharmaceutically Acceptable Reducing Agent

Although the invention elsewhere in this specification is described in relation to glutathione, it is within the scope of the present invention to use in the place of or in addition to glutathione any other pharmaceutically acceptable reducing agent capable of obtaining cysteine protease in reduced active state or maintaining cysteine protease in its reduced active state. In a preferred embodiment of the invention, the pharmaceutically acceptable reducing agent is selected from the group consisting of cysteine, glutathione, a combination of glutathione and glutathione S-transferase, Dithiothreitol (DTT), cysteamine, thioredoxin, N-acetyl-L-cysteine (NAC), alpha-lipoic acid, 2-mercaptoethanol, 2-mercaptoethanesulfonic acid, mercapto-propionyglycine, tris(2-carboxyethyl)phophine (TCEP) and combinations thereof.

### Glutathione S-transferase (GST)

Glutathione S-transferases is a family of enzymes comprising a long list of cytosolic, mitochondrial and microsomal proteins that are capable of multiple reactions with a multitude of substractes, both endogenous and xenobiotic. Glutathione S-transferase catalyses the conjugation of reduced glutathione (GSH) via the sulfhydryl group, to electrophilic centers on a wide variety of substrates. In this process GSH is converted into oxidized glutathione (GSSG).

The glutathione S-transferase of the vaccine composition of the invention may any known naturally occurring glutathione S-transferase or a mutant thereof having the same enzymatic activity. The naturally occurring glutathione S-transferase may originate from any biological organisms containing such enzyme. Glutathione S-transferase is present in a high number of biological organisms, including mammals in general, fish, insects, plants etc. Specific examples of such organisms are the House Dust Mite Dermaphagoides pteronyssinus (Der p) and humans.

The glutathione S-transferase of the vaccine composition of the invention may be obtained by any conventional method therefore, including recombinant techniques and purification from biological materials. The glutathione S-transferase of the invention may be in the form of an extract, a purified fraction of an extract, a modified protein, a recombinant protein or a mutant of a recombinant protein with at least some level of retained enzymatic activity. In a preferred embodiment of the invention, the glutathione S-transferase is in the form of an extract, a purified fraction of an extract or a recombinant protein.

A particular example of a glutathione S-transferase is the glutathione S-transferase from Dermaphagoides pteronyssinus, Der p 8. Like Der p 1 it is found in the faecal pellets from the Der p. Der p 8 has a molecular mass of 25589 Da and is a protein of 219 amino acids.

As a certain level of glutathione is found in i.a. humans, inclusion of glutathione transferase in the vaccine composition will have an effect although the vaccine composition does not contain any glutathione. Likewise, as a certain level of glutathione S-transferase is found in i.a. humans, inclusion of glutathione in the vaccine composition will have an effect although the vaccine composition does not contain any glutathione S-transferase.

### Cysteine Protease in Oxidised Inactive Form

When the cysteine protease used in the vaccine composition of the invention is in oxidised inactive form, the vaccine composition may further include any pharmaceutically acceptable oxidising agent capable of obtaining cysteine protease in oxidised inactive state or maintaining cysteine protease in its oxidised inactive state. In a preferred embodiment of the invention, the pharmaceutically acceptable oxidising agent is selected from the group consisting of glutathione disulfide (GSSG), cystine and cystamine.

### Mucosal Administration

The mucosa to which the allergy vaccine composition is administered may be any suitable mucosa, and the administration includes oral (via the mucosa of the digestive system), nasal, vaginal, sublingual, ocular, rectal, urinal, intramammal, pulmonal, otolar (i.e. via the ear) and buccal administration, preferably buccal or sublingual administration (oromucosal administration). The allergy vaccine composition may be in the form of a spray, an aerosol, a mixture, a suspension, a dispersion, an emulsion, a gel, a paste, a syrup, a cream, an ointment, implants (ear, eye, skin, nose, rectal, and vaginal), intramammary preparations, vagitories, suppositories, or uteritories.

It has been speculated that it is preferable to carry out a mucosal administration of a vaccine via the mucosa, which is subject to the natural exposure to the allergen. Accordingly, for allergies to airborne mucosal antigenic agents, it is preferred to use administration via the respiratory system, preferably an oromucosal administration.

In one embodiment of the invention, the subject is subjected to a vaccination protocol comprising daily administration of the vaccine. In another embodiment of the invention the vaccination protocol comprises administration of the vaccine every second day, every third day or every fourth day. For instance, the vaccination protocol comprises administration of the vaccine for a period of more than 4 weeks, preferably more than 8 weeks, more preferably more than 12 weeks, more preferably more than 16 weeks, more preferably more than 20 weeks, more preferably more than 24 weeks, more preferably more than 30 and most preferably more than 36 weeks.

The period of administration may a continuous period. Alternatively, the period of administration is a discontinuous period interrupted by one or more periods of non-administration. Preferably, the (total) period of non-administration is shorter than the (total) period of administration.

In a further embodiment of the invention, the vaccine is administered to the patient once a day. Alternatively, the vaccine is administered to the patient twice a day. The vaccine may be a uni-dose vaccine.

### Oromucosal Administration

The oromucosal administration may be carried out using any available oromucosal administration formulation, including a solution, a suspension, fast dispersing dosage forms, drops and lozenges.

In a preferred embodiment of the invention, sublingual immunotherapy (SLIT) is used, in which case fast dispersing dosage forms, drops and lozenges are preferred formulations.

Examples of fast dispersing dosage forms are those disclosed in U.S. Pat. No. 5,648,093, WO 00/51568, WO 02/13858, WO99/21579, WO 00/44351, U.S. Pat. No. 4,371,516 and EP-278 877, as well as WO 2004/047794 and WO 2004/075875 filed in the assignee name of ALK-Abelló A/S. Preferred fast dispersing dosage forms are those produced by freeze-drying. Preferred matrix forming agents are fish gelatine and modified starch.

Allergy vaccines in the form of an aqueous solution or a fast-dispersing tablet, cf. WO 04/047794, are particularly suitable for buccal and sublingual administration.

Classical incremental dosage desensitisation, where the dose of allergen in the vaccine composition is increased to a certain maximum, may be used in the present invention. The preferred potency of a unit dose of the vaccine composition is from 150-1000000 SQ-u, more preferred the potency is from 500-500000 SQ-u and more preferably the potency is from 1000-250000 SQ-u, even more preferred 1500-125000 SQ-u, most preferable 1500-75000 SQ-u.

In another embodiment of the invention the vaccine composition is a repeated mono-dose, preferably within the range of 1500-125000 SQ-u, more preferably 1500-75000 SQ-u.

The amount of allergen, which corresponds to a given level of potency, varies strongly depending on the allergen specie. In a further embodiment of the invention the concentration of major allergen in a mono-dose intended for daily administration is from 0.05 to 50 μg, more preferably from 0.05 μg to 30 μg, more preferably from 0.06 μg to 25 μg, more preferably from 0.07 μg to 20 μg, more preferably from 0.08 μg to 15 μg, more preferably from 0.09 μg to 10 μg and most preferably from 0.1 μg to 7 μg.

The allergy vaccine composition used in the method of the invention may be in the form of any formulation suitable for administration to a mucosal surface, including a spray, an aerosol, a mixture, tablets (entero- and not-enterocoated), capsule (hard and soft, entero- and not-enterocoated), a suspension, a dispersion, granules, a powder, a solution, an emulsion, chewable tablets, drops, a gel, a paste, a syrup, a cream, a losenge (powder, granulate, tablets), a fast-dispersing tablet, an instillation fluid, a gas, a vapour, an ointment, a stick, implants (ear, eye, skin, nose, rectal, and vaginal), intramammary preparations, vagitories, suppositories, or uteritories.

It is to be understood that the vaccine of the invention may further comprise additional adjuvants and other excipients suitable for such type of formulation. Such additional adjuvants and excipients are well-known to the person skilled in the art and include i.a. solvents, emulsifiers, wetting agents, plasticizers, colouring substances, fillers, preservatives, viscosity adjusting agents, buffering agents, mucoadhesive substances, and the like. Examples of formulation strategies are well-known to the person skilled in the art.

In a preferred embodiment of the invention, glutathione S-transferase is included in the vaccine composition in such an amount that the molar ratio of glutathione S-transferase to cysteine protease is from 0.001 to 1000, preferably from 0.01 to 100, more preferably from 0.02 to 50, more preferably from 0.05 to 20, more preferably from 0.1 to 10 and most preferably from 0.2 to 5.

In a preferred embodiment of the invention, glutathione is included in the vaccine composition in such an amount that the molar ratio of glutathione to glutathione S-transferase is from 0.1 to 100000, preferably from 1 to 10000, more preferably from 2 to 5000, more preferably from 5 to 2000, more preferably from 10 to 1000 and most preferably from 20 to 500.

### Adjuvant

The allergy vaccine composition may include an adjuvant, which may be any conventional adjuvant, including oxygen-containing metal salts, heat-labile enterotoxin (LT), cholera toxin (CT), cholera toxin B subunit (CTB), polymerised liposomes, mutant toxins, e.g. LTK63 and LTR72, microcapsules, interleukins (e.g. IL-1β, IL-2, IL-7, IL-12, INFγ), GM-CSF, MDF derivatives, CpG oligonucleotides, LPS, MPL, phosphophazenes, Adju-Phos®, glucan, antigen formulation, liposomes, DDE, DHEA, DMPC, DMPG, DOC/Alum Complex, Freund's incomplete adjuvant, ISCOMs®, LT Oral Adjuvant, muramyl dipeptide, monophosphoryl lipid A, muramyl tripeptide, and phospatidylethanolamine.

The oxygen-containing metal salt may be any oxygen-containing metal salt providing the desired effect. In a preferred embodiment, the cation of the oxygen-containing metal salt is selected from Al, K, Ca, Mg, Zn, Ba, Na, Li, B, Be, Fe, Si, Co, Cu, Ni, Ag, Au, and Cr, In a preferred embodiment, the anion of the oxygen-containing metal salt is selected from sulphates, hydroxides, phosphates, nitrates, iodates, bromates, carbonates, hydrates, acetates, citrates, oxalates, and tartrates, and mixed forms thereof. Examples are aluminium hydroxide, aluminium phosphate, aluminium sulphate, potassium aluminium sulphate, calcium phosphate, Maalox (mixture of aluminium hydroxide and magnesium hydroxide), beryllium hydroxide, zinc hydroxide, zinc carbonate, zinc chloride, and barium sulphate.

### Vaccine Adjuvant System

The present invention further relates to an adjuvant system for use in a vaccine for mucosal administration comprising a cysteine protease.

In one embodiment of the adjuvant system according to the invention, the cysteine protease is in a reduced active state. In a particular embodiment of the invention, the adjuvant system further comprises reduced glutathione. In a futher particular embodiment of the invention, the adjuvant system further comprises a glutathione S-transferase. In particular, the glutathione S-transferase is Der p 8.

In a second embodiment of the invention, the cysteine protease is in an oxidised inactive state.

The cysteine protease of the adjuvant system of the invention may be any cysteine protease as defined and described elsewhere in this specification. In a specific embodiment of the invention, the cysteine protease is selected from the group consisting of Aca s 1, Blo t 1, Der f 1, Der p 1, Eur m 1, Gly d 1, Lep d 1, Pso o 1 and Tye p 1. In particular, the cysteine protease allergen is Der p 1.

### Antigen Vaccine Composition

The present invention further relates to an antigen vaccine composition for mucosal administration comprising an antigen and an adjuvant system according to the invention. The antigen vaccine composition of the invention is suitable for vaccination against any disease caused by the antigen of the composition.

### Antigen

Allergen:

In one embodiment of the invention, the antigen of the antigen vaccine composition is an allergen.

In one embodiment of the invention, the allergen is an allergen, which comes into contact with the mucosa of the subject, including the mucosa of the respiratory system, the mucosa of the digestive system, the rectal mucosa and the genital mucosa.

The allergen according to the present invention may be any naturally occurring protein that has been reported to induce allergic, i.e. IgE mediated reactions upon their repeated exposure to an individual. Examples of naturally occurring allergens include pollen allergens (tree-, herb, weed-, and grass pollen allergens), insect allergens (inhalant, saliva and venom allergens, e.g. mite allergens, cockroach and midges allergens, hymenopthera venom allergens), animal hair and dandruff allergens (from e.g. dog, cat, horse, rat, mouse etc.), and food allergens. Important pollen allergens from trees, grasses and herbs are such originating from the taxonomic orders of Fagales, Oleales, Pinales and platanaceae including i.a. birch (Betula), alder (Alnus), hazel (Corylus), hornbeam (Carpinus) and olive (Olea), cedar (Cryptomeria and Juniperus), Plane tree (Platanus), the order of Poales including i.a. grasses of the genera Lolium, Phleum, Poa, Cynodon, Dactylis, Holcus, Phalaris, Secale, and Sorghum, the orders of Asterales and Urticales including i.a. herbs of the genera Ambrosia, Artemisia, and Parietaria. Other important inhalation allergens are those from house dust mites of the genus Dermatophagoides and Euroglyphus, storage mite e.g Lepidoglyphys, Glycyphagus and Tyrophagus, those from cockroaches, midges and fleas e.g. Blatella, Periplaneta, Chironomus and Ctenocepphalides, and those from mammals such as cat, dog and horse, venom allergens including such originating from stinging or biting insects such as those from the taxonomic order of Hymenoptera including bees (superfamily Apidae), wasps (superfamily Vespidea), and ants (superfamily Formicoidae). Important inhalation allergens from fungi are i.a. such originating from the genera Alternaria and Cladosporium.

In a particular embodiment of the invention the allergen is Bet v 1, Aln g 1, Cor a 1 and Car b 1, Que a 1, Cry j 1, Cry j 2, Cup a 1, Cup s 1, Jun a 1, Jun a 2, jun a 3, Ole e 1, Lig v 1, Pla l 1, Pia a 2, Amb a 1, Amb a 2, Amb t 5, Art v 1, Art y 2 Par j 1, Par j 2, Par j 3, Sal k 1, Ave e 1, Cyn d 1, Cyn d 7, Dac g 1, Fes p 1, Hol l 1, Lol p 1 and 5, Pha a 1, Pas n 1, Phl p 1, Phl p 5, Phl p 6, Poa p 1, Poa p 5, Sec c 1, Sec c 5, Sor h 1, Der f 1, Der f 2, Der p 1, Der p 2, Der p 7, Der m 1, Eur m 2, Gly d 1, Lep d 2, Blo t 1, Tyr p 2, Bla g 1, Bla g 2, Per a 1, Fel d 1, Can f 1, Can f 2, Bos d 2, Equ c 1, Equ c 2, Equ c 3, Mus m 1, Rat n 1, Apis m 1, Api m 2, Ves v 1, Ves v 2, Ves v 5, Dol m 1, Dil m 2, Dol m 5, Pol a 1, Pol a 2, Pol a 5, Sol i 1, Sol i 2, Sol i 3 and Sol i 4, Alt a 1, Cla h 1, Asp f 1, Bos d 4, Mal d 1, Gly m 1, Gly m 2, Gly m 3, Ara h 1, Ara h 2, Ara h 3, Ara h 4, Ara h 5 or shufflant hybrids from Molecular Breeding of any of these.

In a preferred embodiment of the invention the allergen is grass pollen allergen or a dust mite allergen or a ragweed allergen or a cedar pollen or a cat allergen or birch allergen.

In yet another embodiment of the invention the allergen is a combination of at least two different types of allergens either originating from the same allergic source or originating from different allergenic sources e.g. grass group 1 and grass group 5 allergens or mite group 1 and group 2 allergens from different mite and grass species respectively, weed antigens like short and giant ragweed allergens, different fungis allergens like alternaria and cladosporium, tree allergens like birch, hazel, hornbeam, oak and alder allergens, food allergens like peanut, soybean and milk allergens.

The allergen incorporated into the antigen vaccine composition of the invention may be in the form of an extract, a purified allergen, a modified allergen, a recombinant allergen or a mutant of a recombinant allergen. An allergenic extract may naturally contain one or more isoforms of the same allergen, whereas a recombinant allergen typically only represents one isoform of an allergen. In a preferred embodiment the allergen is in the form of an extract. In another preferred embodiment the allergen is a recombinant allergen. In a further preferred embodiment the allergen is a naturally occurring low IgE-binding mutant or a recombinant low IgE-binding mutant.

Allergens may be present in equi-molar amounts or the ratio of the allergens present may vary preferably up to 1:20.

Infection:

In a second embodiment of the invention, the antigen of the antigen vaccine composition is a microbial antigen.

The microbial antigen may be an antigen, which comes into contact with the mucosa of the subject, including the mucosa of the respiratory system, the mucosa of the digestive system, the rectal mucosa and the genital mucosa.

In a particular embodiment of the invention, the microbial antigen is a virus, a bacterium, a fungus, a parasite or any part thereof.

Examples of microbial antigens are Vibrio species, Salmonella species, Bordetella species, Haemophilus species, Toxoplasmosis gondii, Cytomegalovirus, Chlamydia species, Streptococcal species, Norwalk Virus, Escherischia coli, Helicobacter pylori, Helicobacter fells, Rotavirus, Neisseria gonorrhae, Neisseria meningiditis, Adenovirus, Epstein Barr Virus, Japanese Encephalitis Virus, Pneumocystis carini, Herpes simplex, Clostridia species, Respiratory Syncytial Virus, Klebsielia species, Shigella species, Pseudomonas aeruginosa, Parvovirus, Campylobacter species, Rickettsia species, Varicella zoster, Yersinia species, Ross River Virus, J. C. Virus, Rhodococcus equi, Moraxella catarrhalis, Borrella burgdorferi, Pasteurella haemolytica, poliovirus, influenza virus, Vibrio cholerae and Salmonella enterica serovar Typhi.

Further examples of microbial antigens are those, which prevent or reduce the symptoms of the following diseases: Influenza, Tuberculosis, Meningitis, Hepatitis, Whooping Cough, Polio, Tetanus, Diphtheria, Malaria, Cholera, Herpes, Typhoid, HIV, AIDS, Measles, Lyme disease, Travellers Diarrhea, Hepatitis A, B and C, Otitis Media, Dengue Fever, Rabies, Parainfluenza, Rubella, Yellow Fever, Dysentery, Legionnaires Disease, Toxoplasmosis, Q-Fever, Haemorrhegic Fever, Argentina Haemorrhagic Fever, Caries, Chagas Disease, Urinary Tract Infection caused by E. coli, Pneumoccoccal Disease, Mumps, and Chikungunya.

### Definitions

In connection with the present invention the following terms and expressions are used:

The expression “a papain-like cysteine protease of clan CA or of clan CC” means the proteases defined and listed as belonging to clans CA and CC in Handbook of Proteolytic Enzymes edited by Alan 3. Barrett et al., Academic Press, 1998, as well as presently unknown proteases belonging to the CA and CC clans.

The term “allergen” means any compound capable of eliciting allergy as defined below.

The term “allergy” means any type of hypersensitivity reaction to an environmental allergen mediated by immunological mechanisms, including Type I-IV hypersensitivity reactions, including allergic rhinitis, asthma and atopic dermatitis.

The term “allergy vaccine composition” means vaccine composition for treating or preventing allergy.

The term “treating” means partly or wholly curing or alleviating symptoms, or inhibiting causes of symptoms.

The term “preventing” means any type of prophylactic treatment, including partly or wholly preventing or inhibiting the development of symptoms or the development of causes of symptoms.

The term “oromucosal administration” refers to a route of administration where the dosage form is placed under the tongue or anywhere else in the oral cavity (buccal administration) to allow the active ingredient to come in contact with the mucosa of the oral cavity or the pharynx of the patient in order to obtain a local or systemic effect of the active ingredient. An example of an oromucosal administration route is sublingual administration.

The term “sublingual administration” refers to a route of administration, where a dosage form is placed underneath the tongue in order to obtain a local or systemic effect of the active ingredient.

The term “SQ-u” means SQ-Unit: The SQ-Unit is determined in accordance with ALK-Abelló A/S's “SQ biopotency”-standardisation method, where 100,000 SQ units equal the standard subcutaneous maintenance dose. Normally 1 mg of extract contains between 100,000 and 1,000,000 SQ-Units, depending on the allergen source from which they originate and the manufacturing process used.

The precise allergen amount can be determined by means of immunoassay i.e. total major allergen content and total allergen activity.

### EXAMPLE

### Materials and Methods

Purification of nDer p 1

The purification of nDer p 1 was done in two consecutive affinity chromatographic steps. 534 mg of D. pteronyssinus was dissolved in approximately 9.5 ml of binding buffer (PBS, pH 7.2) and filtered through a 0.45 μm cut-off filter (Millex®-HV, MILLIPORE) to remove non soluble compounds.

A 7 ml column was used containing sepharose with a monoclonal antibody against Der p 1 (4C1, Indoor Biotechnologies). The column was connected to an ÄKTA prime (Amersham Pharmacia biotech product). Prior to loading the sample into the loop and injecting it onto the column, the column was equilibrated in binding buffer.

After applying the entire sample, the column was washed thoroughly with 3 column volumes of binding buffer. The protein was then eluted by applying a linear NaCl/glycin gradient to the elution buffer (0.1 M glycin pH 11, 0.5 NaCl) for 5 minutes, which caused a release of Der p 1 from the antibody.

The elution was collected in 1 ml fractions. To neutralize the alkaline elutate 200 μl of 1 M NaAcetat (pH 5.0) was added to all the collection tubes. The protein elution was followed by monitoring the absorption at 280 nm (A280) of the elution fractions. The fractions containing the elution peak were then pooled.

Subsequently, the pool containing Der p 1 was concentrated to ˜2 ml using a 5 kDa cut-off spin filter (MILLIPORE, Amicon®Ultra, Centrifugal Filter Devices).

Previous studies have shown that the serine protease Der p 3 co-elute with Der p 1 from the 4C1-sepharose column. Therefore the sample containing the elution peak was subjected to a new affinity chromatography on an agarose column containing SBTI (Soya bean Trypsin inhibitor-agarose, SIGMA-ALDRICH). SBTI is an inhibitor of serine proteases.

The buffers used for this were the same as for the 4C1-sepharose column. Der p 1 was expected to be in the flow-through as the column only binds Der p 3. The fractions containing the non-bound protein were identified by measuring the A280 and pooled.

During the chromatography process the column and all the buffers were kept at 4° C.

Purification of nDer p 8

The purification of nDer p 8 was done in one affinity chromatography step using a GSTrap HP, 1 ml Hi Trap affinity column (Amersham Biosciences). The manufacturers' indications were followed.

486 mg extract from D. pteronyssinus was dissolved in 9.6 ml of binding buffer (PBS, pH 7.2) and passed through a 0.45 μm cut-off filter (Millex®-HV, MILLIPORE). The column retained the nDer p 8, which was then eluted (elution buffer: 50 mM Tris-HCl, 10 mM reduced glutathione, pH 8.0). The protein elution was followed by measuring the A280 of the fractions, and those containing the elution peak were pooled.

GSH was removed by dialyses (see procedure further below) and the sample was divided in aliquots and kept at −20° C.

### UV/VIS Absorption Spectroscopy

The absorbance at 280 nm was measured repeatedly during the purifications of the proteins, nDer p 1 and Der p 8, in order to identify which fractions contained the proteins. The UV/VIS absorption spectra between 220 nm and 350 nm of the final preparations of purified nDer p 1 or nDer p 8 were recorded in order to calculate the protein concentration, using the Lambert-Beers law.

The measurements were done with a PerkinElmer™ instrument (model: Lambda 800 UV/VIS Spectrometer) and the cuvette used had a path length of 1 cm (Hellma®, quartz cuvette).

Prior to the readings the spectrophotometer was zeroed against a blank sample. When measuring the absorbance of nDer p 1 the elution buffer was used as reference. For nDer p 8 the reference was one of the eluted fractions which contained no protein. Between each reading the cuvette was cleaned thoroughly with 2% Helmax (helmanex) solution and milliQ water and dried with high air pressure.

Dialysis of nDer p 8

In order to investigate whether the activity of Der p 1 is dependent of GSH and if nDer p 8 could catalyse the process, GSH was removed from the nDer p 8 pool. This was done by applying dialysis cassette (Slide-A-Lyser® Dialysis 10K, PIERCE product). The membrane was hydrated by placing the cassette in a buoy and then placing it in milliQ water for 30 seconds. Der p 8 preparation was then injected by a syringe removing excess air. Afterwards the cassette was placed in 1 litre of the dialysis buffer (PBS, pH 7.2) and placed on magnetic stir for circulation. After 2½ hours the cassette was placed in another litre of dialysis buffer. Further 2½ hour later the preparation was removed from the cassette, the UV/VIS absorption was measured and the concentration of Der p 8 calculated. The Der p 8 pool was then divided into aliquots and kept at −20° C.

### SDS-PAGE

13 μμl of the sample of interest were mixed with 5 μl sample buffer×4 (NuPAGE®, Invitrogen). Depending on whether the SDS-PAGE should be performed under reduced or non-reduced conditions, samples were prepared accordingly by applying 2 μl reducing agent (NuPAGE®, Invitrogen). In non-reduced conditions the reducing agent was replaced by 2 μl milliQ water.

The samples were then heated at 70° C. for 10 minutes and subsequently spun for ten seconds.

40 ml Running buffer×20 (NuPAGE®, Invitrogen) were mixed with 800 μl milliQ water to prepare the electrophoresis buffer. A gel (NuPAGE® 10% Bis-Tris gel, Invitrogen) was placed in the XCell Surelock electrophoresis cell (Invitrogen) and 600 ml of the electrophoresis buffer was inserted into the outer chamber. 0.5 ml of antioxidant (NuPAGE®, Invitrogen) was added to the remaining 200 ml of electrophoresis buffer, and this solution was poured into the inner chamber.

After a molecular weight arker (SeeBlue Plus 2 Pre-Stained Standard, Invitrogen) and 15 μl of each sample were loaded into the gel, the electrophoresis was run for 40 minutes at a constant voltage of 200 V.

### SDS-PAGE Gel Staining

After the electrophoresis the gels were stained with silver staining according to the following protocol: Fixation solution: 30 minutes; Incubation solution (50 ml incubation buffer+260 μl 25% glutaraldehyd): 30 minutes; Washed in milliQ water: 3×10 minutes; Silver solution (50 ml silver solution+10 μl 37% formaldehyde): 40 minutes; Washed in milliQ water: Less than one minute; Developer solution (50 ml developer solution+5 μl 37% formaldehyde): Until the bands become visible; Reaction is stopped with stopping solution: 10 minutes; Washed in milliQ water; 2×5 minutes; Stored in storage buffer.

Fixation solution: 40% Ethanol, 10% acetic acid

Incubation solution: 30% Ethanol, 0.83 M Sodium acetate, 8 mM Sodium thiosulphate

Silver solution: 5.9 mM Silver nitrate

Developing solution: 0.24 M Sodium carbonate

Stop solution: 39 mM EDTA

### Western Blot

In order to verify the identity of the protein in the samples applied in the SDS-PAGE, a gel was subjected to Western blot. The proteins separated in the gel by electrophoresis were transferred to a PVDF membrane by Western blotting. The entire process was performed at room temperature.

Four Blotting Pads were first washed in milliQ water and then soaked in a transfer buffer (Transfer Buffer (20×) (NuPAGE®), 96% Ethanol (Spiritus Fortis DLS), Antioxidant (NuPAGE®), and milliQ water). Air was pressed out of the Blotting Pads and two were placed in the XCell II Blot Module. The filter paper was also soaked in transfer buffer prior to its application on the XCell. The gel from the electrophoresis was placed on top of the filter paper. A PVDF membrane (Invitrogen) was soaked for 30 seconds in ethanol, then rinsed in milliQ water and was finally placed on the gel after lying in a transfer buffer for a couple of minutes. Another wet piece of filter paper was placed on top of the membrane and then two more Blotting Pads were added. The apparatus was assembled and placed in an XCell Surelock electrophoresis cell (Invitrogen). The inner chamber was filled with transfer buffer and the outer chamber was filled with milliQ water. The blotting was carried out at 30 V for 1 hour.

The membrane was then treated and stained to reveal the presence of Der p 1, according to the following immunoblotting procedure: Blocking (washing buffer+2% Tween): Not more than 2 minutes; Washing with washing buffer: 5 minutes; Primary antibody (rabbit α-Der p 1, ALK-Abelló): 1½ hours; Washing with washing buffer: 3×5 minutes; Secondary antibody (pig α-rabbit, Dako); 1 hour; Washing with washing buffer: 3×5 minutes; Visualisation (1 tablet BCIP/NBT (Sigma) is dissolved in 10 ml milliQ water): Until bands become visible; The reaction is stopped with milliQ water; The membrane dries on filter paper.

Washing buffer: 50 mM Tris, 150 mM Sodium chloride

### Assays of Proteolytic Activity

The enzymatic activity of nDer p 1 was evaluated using the peptide substrate Z-LLE linked to the fluorescent group AMC. AMC is quenched when linked to the peptide and only shows very little fluorescence. When Der p 1 is added, it cleaves the bond between AMC and the peptide sequence and the fluorescence increases considerably. Therefore the product formation was evaluated in terms of increase in fluorescence.

All activity assays were performed on a Spectra Max instrument (GeminiXS Molecular Devises) at a temperature of 37° C. The fluorescence was measured using an excitation wavelength (λex) of 350 nm and emission wavelength (λem) of 450 nm. The Spectra Max instrument was set to automix before the first read and in between reads. The enzymatic activity was measured in RFU, relative fluorescent units. Using the software SoftMax® PRO 4.3 LS, the initial velocity of the reaction (V0) was estimated from the maximal slope of the progress curve.

Stock solutions of assay buffer (50 mM Tris (Sigma), 5 mM EDTA (Fluka)) and 1 M DTT (Merck) were made once and kept at +5° C. and −20° C. respectively. DTT was kept in aliquots of 55 μl, and when necessary DTT was added to the assay buffer just before use.

The total volume applied for each well in the micro titer plate (Corning, 96-well non-binding black polystyrene plate) amounted to 200 μl. The reaction was always started by the addition of 50 μl of 0.1 mM or 1 mM substrate diluted in assay buffer, and the fluorescence was measured continuously for 20-90 minutes.

An enzymatic assay in the presence or absence of the cysteine protease specific inhibitor E64 was used in order to verify the identity of the purified protein as cysteine protease. One aliquot of nDer p 1 was pre-incubated for 1 hour at 37° C. with 25 μM E64 dissolved in assay buffer, while another aliquot was not. Afterwards the substrate was added at 0.1 mM concentration. The fluorescence was measured for 20 minutes. The concentration of nDer p 1 in the assay was 2.6 μM.

It was tested whether glutathione (GSH) could activate nDer p 1, and whether the activation was dose-dependent. The activity of 1.3 μM nDer p 1 was determined in the presence of different GSH concentrations of 0.5 mM, 1.0 mM, 5.0 mM, 10.0 mM and 25.0 mM. As a control, the activity was also measured in the presence of 5 mM DTT. The fluorescence was measured for 90 minutes.

It was evaluated whether nDer p 8, as a glutathione S-transferase, could catalyse the activation of nDer p 1 by GSH. In this assay, nDer p1 was kept at 1.3 μM and the GSH concentration at 100 μM (physiological concentration in the airways). nDer p 8 was added at four different concentrations: 0.4 μM, 0.6 μM, 0.8 μM and 1.2 μM. The last two components to be added to the well were GSH quickly followed by the substrate. The reaction was followed during 90 minutes.

### Results

nDer p 1 Purification

nDer p 1 was purified in two affinity chromatographic steps. In the first a 4C1-sepharose column was used. The fractions in the elution peak were collected and concentrated. After the concentration the preparation was subjected to the second chromatography on a SBTI-agarose column. nDer p 1 flows through the column without binding and comes out in two peaks. The fractions from each of the peaks were gathered in two separate pools, Pool 1 and Pool 2.

The purity and identity of the purified proteins were evaluated after SDS-PAGE by silver staining or Western blot using a polyclonal mono-specific Der p 1 antibody, respectively.

Both pools contained a major band at ˜25 kDa which is recognised by the antibody as Der p 1. The antibody also recognises some bands of lower molecular weight that probably are degradation fragments of Der p 1. The smear on the SDS-PAGE between approx. 10-16 kDa is not recognised by the antibody and is probably caused by contaminants, or small fragments resulting from the degradation of nDer p 1. A high molecular weight band around 55 kDa is recognised by the antibody and might represent some Der p 1 dimers.

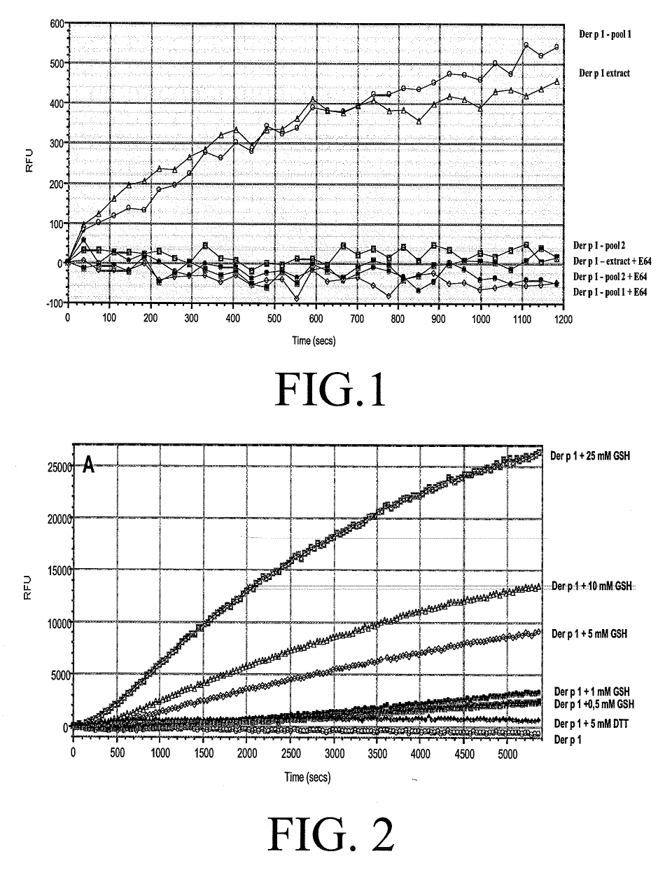

The enzymatic activity of Der p 1 was evaluated in both pools in the presence as well as absence of cysteine protease inhibitor, E64, with DTT as a reducing agent. The first pool showed a clear activity, while the second pool did not show any significant activity. The results are depicted in FIG. 1.

Because Z-LLE-AMC is a specific substrate for cysteine proteases (Willenbrock H and Sierakowska A, 2002, Masther Thesis) and because the activity is completely inhibited by the cysteine protease inhibitor, E64, and Der p 1 is the only cysteine protease reported to be present in D. pteronyssinus faecal particles, these data confirm that the protein purified was Der p 1.

The UV/VIS absorbance spectra of the two pools were measured and the concentrations were calculated from the absorption at 280 nm, using the Lambert-Beer law with a light path length of 1 cm and a molar extinction coefficient of 1.73. The concentrations were 0.259 mg/ml for pool 1 and 0.0943 mg/ml for pool 2.

Purification of nDer p 8

nDer p 8 was purified on a GSTrap HP, Hi Trap affinity column. The SDS-PAGE gel analysis of the fractions corresponding to the elution peak showed only one band with a molecular weight around 25 kDa.

After purification the protein preparation was expected to have the same GSH concentration as the elution buffer; that is 10 mM GSH. In order to test if GSH can activate Der p 1 at the same concentrations found in the airways of the lungs, and if Der p 8 can catalyze this reaction, it is important to remove GSH from the nDer p 8 preparation. This was done by dialysis. Afterwards, the concentration of nDer p 8 was calculated by UV/VIS absorption spectroscopy using extinction coefficient (E^{0.1%})at 280 nm, 1.576 (mg/ml)⁻¹ cm⁻. The final nDer p 8 concentration was 0.121 mg/ml.

Activation of nDer p 1 by GSH

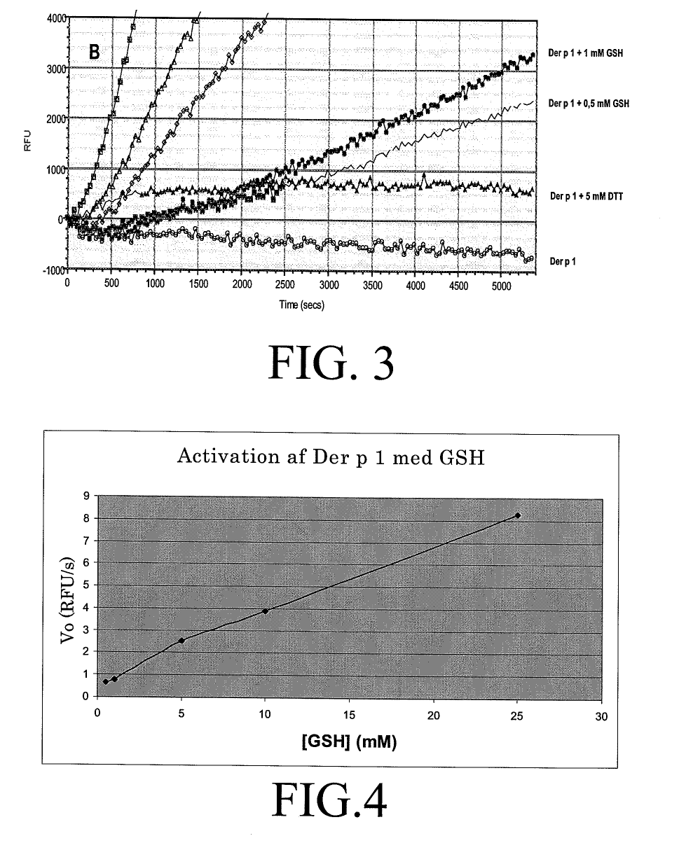

FIGS. 2 and 3 show that nDer p 1 can be activated by GSH and that the activation is indeed dose-dependent. Hence, a larger concentration of GSH results in higher activity and as such there is no activity when GSH is absent, It seems like the activation by GSH is slower than the activation by DTT. In fact, the activation by physiological concentrations of GSH in the airways (0.1-0.5 mM) has a lag time of around 30 minutes (time required to detect enzyme activity). With a concentration of 5.0 mM GSH the activity evolves after approx. 8 minutes, while using 10.0 mM GSH the activity boosts after 6 minutes. At the highest concentration of GSG, which was 25.0 mM, the activity increases notably after only 3 minutes.

FIG. 4 shows that there is an approximate linear correlation between the initial velocity of the activity and the GSH concentration.

Enzymatic activity assays with Der p 8

Knowing that nDer p 1 can be activated by GSH it was pertinent to test if nDer p 8 as a glutathione S-transferase would be able to catalyze the process.

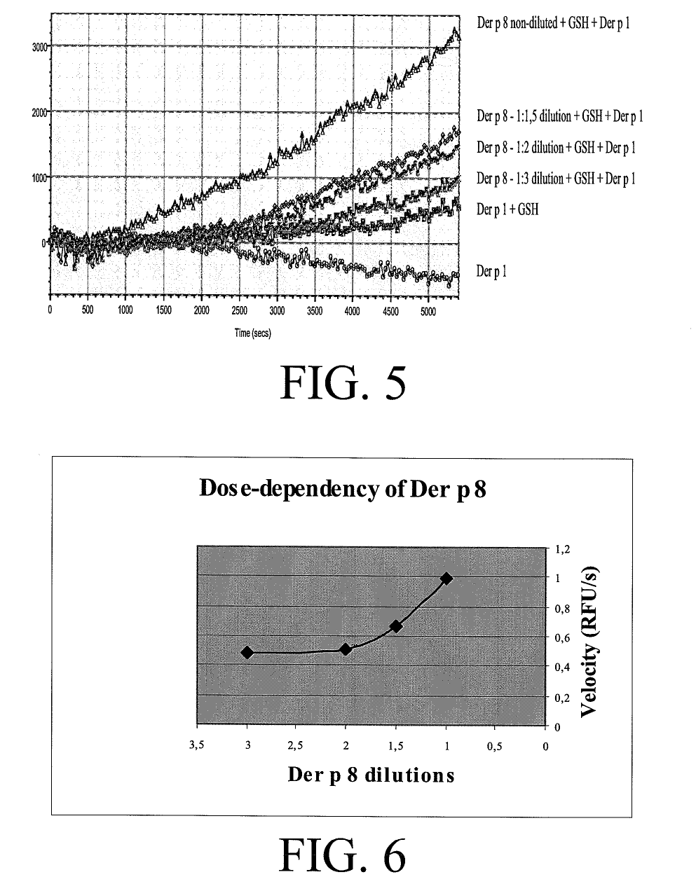

The physiological level of GSH in the human airway epithelial lining fluid is 100 μM-500 μM. The effect of nDer p 8 was tested at the lowest level, 100 μM. FIG. 5 shows the results from the activity assay.

Der p 1 had no activity alone. Even though there was substrate present, it could not be cleaved before Der p 1 was activated by a reducing agent, such as GSH. When Der p 1 was activated by 0.1 mM GSH, the activity was very low and it began after a long lag time of approximately 30 minutes. In the presence of Der p 8 the activity was higher and the lag time shorter. These two effects were dose-dependent. The velocity of the reaction was measured at the very end of the assay. FIG. 6 shows how the velocity increases significantly at higher nDer p 8 concentrations.

### Discussion of the Results

The two allergens from D. pteronyssinus, nDer p 1 and nDer p 8, were purified by affinity chromatography.

After the first purification step, nDer p 1 was applied to a SBTI column to remove contaminating serine proteases (Der p 3) yielding two pools containing nDer p 1, as verified by Western blotting. Thus, the purification yielded two pools with a nDer p 1 concentration of 0.259 mg/ml and 0.094 mg/ml, respectively.

Since the SBTI column will only bind Der p 3, Der p 1 should run right through. Therefore, it was expected to recover nDer p 1 in one peak. There might be different reasons why Der p 1 elutes from the SBTI agarose column in two peaks. The column was packed by hand, so there is a possibility that some irregularities are generated in the process of packaging the column. If the column is heterogeneous, nDer p 1 molecules could have difficulties getting through the more dense areas in the column and as a result, they will be eluted in different tempi. Another possible explanation is the existence of two different types of nDer p 1 molecules, one of them been able to interact somehow with the column, resulting retarded in their elution

Apart from verifying that the major protein purified was nDer p 1, the Western blot also showed some degradation fragments of nDer p 1, as well as other minor proteins of low molecular weight. These are contaminants, meaning unrecognized by the polyclonal anti-Der p 1 antibody used. Since Der p 1 is a protease, it is possible that it has cleaved itself by autoproteolysis—thus, resulting in the bands with the molecular weights between 16 and 24 kDa. Some extent of autoproteolysis is likely to occur even if Der p 1 is kept cool at all times.

The first enzymatic activity assay showed that there was activity in the nDer p 1 pool 1 and that it was entirely inhibited by E64. The impurities in the nDer p 1 pool were therefore not causing any undesired contributions to the activity of nDer p 1. Pool 2 did not show any activity. Perhaps the low nDer p 1 concentration does not allow any detection of the activity or perhaps pool 2 contains denatured nDer p 1. This last possibility could also justify why this nDer p 1 elutes from the SBTI agarose column in a different peak than the active nDer p 1.

nDer p 8 was purified, and an SDS-PAGE of this purification followed by silver staining showed only one band with the right molecular weight—approx. 25 kDa. Since the purified protein showed the expected gluthathione S-transferase activity the protein was very likely to be in fact nDer p 8.

GSH is able to activate nDer p 1, and the activity of nDer p 1 increased in correlation to the increase of GSH concentrations: the higher the GSH concentration, the higher the nDer p 1 activity and the shorter the lag time to get nDer p 1 activation.

It is known that the reduction of proteins by GSH is slow and inefficient, and in the metabolic process where this reduction takes place, the reaction is catalyzed by glutathione S-transferases. Since Der p 8 is described to be a glutathione S-transferase, it was examined whether nDer p 8 could catalyse the activation of nDer p 1 by GSH, making it more efficient. As the physiological level of GSH in human lung airways is between 100 μM and 500 μM, it was decided to examine the effect of nDer p 8 on the nDer p 1 activation by a GSH concentration of 0.1 mM (100 μM). This assay proved that Der p 8 enhances the activation of nDer p 1 by GSH. At the lowest concentrations of nDer p 8 used (0.4 μM) it took over 30 minutes until the activities differentiated themselves from the test without nDer p 8. With 1.2 μM Der p 8 the activity increased after approximately 8 minutes and kept increasing throughout the whole measuring period, which extended to 90 minutes. FIG. 6 shows that the level and velocity of activation of nDer p 1 increased at higher Der p 8 concentrations. In other words, the effect of nDer p 8 on the GSH activation of nDer p 1 is dose-dependent.

Conclusively, the present experiments clearly illustrate that nDer p 1 can be activated by GSH at a physiological level and that the level of activity and lag time were dose-dependent. Furthermore, the results have shown that nDer p 8, which travels together with nDer p 1 in the D. pteronyssinus faecal pellets, is capable of catalysing the process due to its glutathione S-transferase activity. This catalysis is dose-dependent. Moreover, the present experiments have shown that it is possible to prepare reduced active Der p 1 in vitro and to administer a vaccine composition containing reduced active Der p 1 to a mucosal surface in vivo while maintaining the Der p 1 in its reduced active state. Also, the present experiments have shown that the inclusion of GSH and glutahione S-transferase in the vaccine composition will help maintaining the Der p 1 in its reduced active state upon administration.

1. An allergy vaccine composition for mucosal administration comprising a cysteine protease allergen in a reduced active state.

2. A composition according to claim 1, wherein 70% of the cysteine protease allergen, preferably 80%, more preferably 90%, more preferably 95%, more preferably 98% is in a reduced active state.

3. A composition according to claim 1 or 2 further comprising a pharmaceutically acceptable reducing agent.

4. A composition according to claim 3, wherein the pharmaceutically acceptable reducing agent is selected from the group consisting of cysteine, glutathione, a combination of glutathione and glutathione S-transferase, Dithiothreitol (DTT), cysteamine, thioredoxin, N-acetyl-L-cysteine (NAC), alpha-lipoic acid, 2-mercaptoethanol, 2-mercaptoethanesulfonic acid, mercapto-propionyglycine or tris(2-carboxyethyl)phophine (TCEP).

5. A composition according to claim 4, wherein the pharmaceutically acceptable reducing agent is reduced glutathione (GSH).

6. A composition according to any of claims 1-5 further comprising a glutathione S-transferase (GST).

7. A composition according to claim 6, wherein the glutathione S-transferase is Der p 8.

8. An allergy vaccine composition for mucosal administration comprising a cysteine protease allergen in an oxidised inactive state.

9. A composition according to claim 8, wherein 70% of the cysteine protease allergen, preferably 80%, more preferably 90%, more preferably 95%, more preferably 98% is in an oxidised inactive state.

10. A composition according to any of the preceding claims, wherein the cysteine protease allergen is selected from the group consisting of Ale o 1, Aca s 1, Blo t 1, Der f 1, Der p 1, Der m 1, Der s 1, Eur m 1, Gly d 1, Lep d 1, Pso o 1, Sui m 1 and Tyr p 1, in particular Der p 1.

11. A composition according to claim 10, wherein the cysteine protease allergen is Der p 1.

12. A composition according to any of the preceding claims, wherein the mucosal administration is selected from the group consisting of oral (via the mucosa of the digestive system), nasal, vaginal, sublingual, ocular, rectal, urinal, intramammal, pulmonal, otolar (i.e. via the ear) and buccal administration.

13. A composition according to claim 12, wherein the mucosal administration is selected from the group consisting of sublingual and buccal administration.

14. A composition according to any of the preceding claims comprising an adjuvant.

15. An adjuvant system for use in a vaccine for mucosal administration comprising a cysteine protease.

16. An adjuvant system according to claim 15, wherein the cysteine protease is in a reduced active state.

17. An adjuvant system according to claim 15 or 16, wherein the adjuvant system further comprises reduced glutathione.

18. An adjuvant system according to any of claims 15-17, wherein the adjuvant system further comprises a glutathione S-transferase.

19. An adjuvant system according to claim 18, wherein the glutathione S-transferase is Der p 8.

20. An adjuvant system according to claim 15, wherein the cysteine protease is in an oxidised inactive state.

21. An adjuvant system according to any of the claims 15-20, wherein the cysteine protease allergen is selected from the group consisting of Ale o 1, Aca s 1, Blo t 1, Der f 1, Der p 1, Der m 1, Der s 1, Eur m 1, Gly d 1, Lep d 1, Pso o 1, Sui m 1 and Tyr p 1.

22. An adjuvant system according to claim 21, wherein the cysteine protease allergen is Der p 1.

23. An antigen vaccine composition for mucosal administration comprising an antigen and an adjuvant system according to any of claims 15-22.

24. A composition according to 23, wherein the antigen is an allergen.

25. A composition according to claim 24, wherein the antigen is selected from the group consisting of a grass pollen allergen, a dust mite allergen, a ragweed allergen, a cedar pollen, a cat allergen and a birch allergen.

26. A method of preventing or treating allergy in a subject in need thereof comprising administering a vaccine composition according to any claim 1-14 or 24-25 to the subject.
